(19) United States
(12) Patent Application Publication
(10) Pub. No.: US 2003/0086892 A1
(43) Pub. Date: May 8, 2003

(54) Cosmetic compositions comprising polyethylene glycols

(75) Inventors: Peter Klug, Grossostheim (DE); Torsten Henning, Bad Soden (DE)

Correspondence Address: CLARIANT CORPORATION, INTELLECTUAL PROPERTY DEPARTMENT, 4000 MONROE ROAD, CHARLOTTE, NC, 28205, US

(73) Assignee: Clariant GmbH

(21) Appl. No.: 10/272,097

(22) Filed: Oct. 16, 2002

(30) Foreign Application Priority Data
Oct. 16, 2001 (DE) 101 51 046.2

(51) Int. Cl. A61K 7/06; A61K 7/11

(52) U.S. Cl. 424/70.13

(57) ABSTRACT

The invention relates to cosmetic compositions comprising polyethylene glycols of the formula H(OCH₂CH₂)ₙOH, in which n is an integer between 150 and 900. Through the use of the polyethylene glycols, the feel of the skin and the hair, the combability of the hair, and the foaming are improved.

### FIELD OF THE INVENTION

The invention relates to cosmetic compositions comprising polyethylene glycol. Through the use of the polyethylene glycols, the feel of the skin and the hair, the foaming, and the combability of the hair are improved.

### BACKGROUND OF THE INVENTION

Body cleaning and bodycare in two steps is time-consuming. For this reason, compositions with simultaneously cleaning and care action are preferred by many consumers. A large number of cosmetic products attempt to meet this requirement.

U.S. Pat. No. 5,612,307 describes aqueous, liquid body-cleaning compositions which, in addition to the customary surfactants, comprise a care component from the group of silicone oils, fats, oils, waxes, hydrophobic plant extracts, fatty acids, alcohols, esters, lipids and phospholipids.

WO 94/03152 describes shower gels consisting essentially of a surfactant, silicone oil and a cationic polymer.

However, it is hitherto unsatisfactory that care and moisturizing components cannot be incorporated simultaneously in an adequate amount into aqueous cosmetic cleaning compositions.

A further problem consists in the fact that aqueous dispersions comprising surfactant systems and moisturizing and care components separate over the course of time and are thus not very storage-stable.

J. P. Pavlichko et al. describe in HAPPI, Vol. 38, April 2001, pp. 94-99, that high molecular weight polyethylene oxides of the formula H(OCH₂CH₂)ₙOH where n=2,000 to 100,000, i.e. molar masses of about 88,000 to 4,400,000 g/mol, have positive effects in skincare and haircare products. The addition of the high molecular weight polyethylene oxides brought about an improved feel in the skin and hair, an improvement in the foaming and an improved combability of the hair. However, a disadvantage of the high molecular weight polyethylene oxides is that they are difficult to incorporate since they swell very rapidly and form gels.

### SUMMARY OF THE INVENTION

Surprisingly, it has now been found that low molecular weight polyethylene glycols of the formula H(OCH₂CH₂)ₙOH where n is 150 to 900, likewise bring about an improved feel of the skin and hair, an improved combability of the hair and an improved foaming in cosmetic compositions. In this connection, the polyethylene glycols act as foam regulators, via the addition of which it is possible to control the creaminess, the ability to be washed off and the bubble size of the foam. With the use of the low molecular weight polyethylene glycols, it is of great advantage, in contrast to the high molecular weight polyethylene oxides described in HAPPI, Vol 38, April 2001, pp. 94-99, that they are easy to incorporate.

The invention therefore provides cosmetic compositions comprising polyethylene glycols of the formula H(OCH₂CH₂)ₙOH, in which n is an integer between 150 and 900.

Preference is given to polyethylene glycols of the formula H(OCH₂CH₂)ₙOH, in which n is an integer between 180 and 800, particularly preferably between 240 and 800, especially preferably between 300 and 800.

The cosmetic compositions preferably comprise, based on the finished compositions, 0.1 to 50% by weight, preferably 0.5 to 10% by weight, especially preferably 1 to 4% by weight, of the polyethylene glycols.

In a particular embodiment, the cosmetic compositions additionally comprise cationic guar gum polymers, as described in WO 97/26854. Surprisingly, it has been shown that the simultaneous use of the cationic guar gum polymers significantly increases the affinity of the polyethylene glycols on the skin.

The cationic guar gum derivatives are obtainable by reacting the hydroxyl groups of the polygalactomannan backbone with reactive quaternary ammonium compounds.

Preferred cationic guar gum polymers are those described in WO 97/26854, the entire contents of which is hereby expressly included in the present application.

Particularly preferred cationic guar gum polymers are guar hydroxypropyl-trimethylammonium chlorides.

The molecular weight of the cationic guar gum polymers is preferably 2,000 to 3,000 000 g/mol.

The finished cosmetic compositions preferably comprise 0.01 to 1.0% by weight, particularly preferably 0.02 to 0.4% by weight, of cationic guar gum polymers.

The cosmetic compositions according to the invention are preferably shampoos, preferably hair shampoos, rinses, conditioners, cream rinses, creams, ointments, gels, shower gels, shower preparations and foam baths.

### DETAILED DESCRIPTION OF THE INVENTION

A particular advantage of the polyethylene glycols with regard to the formation possibilities consists in the fact that they can be combined without problems with all customary anionic, cationic, zwitterionic, nonionic and amphoteric surfactants in aqueous or aqueous-alcoholic media.

In this connection, the total amount of surfactants, based on the finished cosmetic compositions, is preferably between 5 and 70% by weight, particularly preferably between 10 and 40% by weight, especially preferably between 12 and 35% by weight.

As anionic surfactants, preference is given to (C₁₀-C₂₀)-alkyl and alkylene carboxylates, alkyl ether carboxylates, fatty alcohol sulfates, fatty alcohol ether sulfates, alkylamide sulfates and sulfonates, fatty acid alkylamide polyglycol ether sulfates, alkane sulfates, alkanesulfonates and hydroxyalkanesulfonates, olefinsulfonates, acyl esters of isethionates, α-sulfo fatty acid esters, alkylbenzenesulfonates, alkylphenol glycol ether sulfonates, sulfosuccinates, sulfosuccinic mono- and diesters, fatty alcohol ether phosphates, protein fatty acid condensation products, alkyl monoglyceride sulfates and sulfonates, alkyl glyceride ether sulfonates, fatty acid methyltaurides, fatty acid sarcosinates, sulforicinoleates, amphoacetates or glycinates and/or acyl glutamates. The anionic surfactants are preferably used in the form of their water-soluble or water-dispersible salts, e.g. as sodium, potassium, magnesium, ammonia, mono-, di- and triethanolammonium or alkylammonium salts.

The proportion by weight of the anionic surfactants is, based on the finished compositions according to the invention, preferably 0.1 to 50% by weight, particularly preferably 7 to 30% by weight, especially preferably 9 to 18% by weight.

As cationic surfactants, preference is given to quaternary ammonium salts, preferably di(C₁₀-C₂₄)alkyldimethylammonium chloride and bromide, particularly preferably di(C₁₂-C₁₈)alkyldimethylammonium chloride and bromide; (C₁₀-C₂₄)alkyldimethylethylammonium chloride and bromide; (C₁₀-C₂₄)alkyltrimethyl-ammonium chloride and bromide, preferably cetyltrimethylammonium chloride and bromide and (C₂₀-C₂₂)alkyltrimethylammonium chloride and bromide; (C₁₀-C₂₄)alkyldimethylbenzylammonium chloride and bromide, preferably (C₁₂-C₁₈)alkyldimethylbenzylammonium chloride; N-(C₁₀-C₁₈)alkylpyridinium chloride and bromide, preferably N-(C₁₂-C₁₆)alkylpyridinium chloride and bromide; N-(C₁₀-C₁₈)alkylisoquinolinium chloride, bromide and monoalkylsulfate; N-(C₁₂-C₁₈)-alkylpolyoylaminoformylmethylpyridinium chloride; N-(C₁₂-C₁₈)alkyl-N-methylmorpholinium chloride, bromide and monoalkyl sulfate; N-(C₁₂-C₁₈)alkyl-N-ethylmorpholinium chloride, bromide and monoalkyl sulfate; (C₁₆-C₁₈)alkyl-pentaoxethylammonium chloride; diisobutyl-phenoxyethoxyethyldimethylbenzylammonium chloride; salts of N,N-diethylaminoethylstearylamide and -oleylamide with hydrochloric acid, acetic acid, lactic acid, citric acid, and phosphoric acid; N-acylaminoethyl-N,N-diethyl-N-methyl-ammonium chloride, bromide and monoalkyl sulfate; and/or N-acylaminoethyl-N,N-diethyl-N-benzylammonium chloride, bromide and monoalkyl sulfate, where the acyl radicals are preferably stearoyl or oleoyl radicals.

The proportion by weight of the cationic surfactants, based on the finished compositions according to the invention, is preferably 1 to 10% by weight, particularly preferably 2 to 7% by weight, especially preferably 3 to 5% by weight.

As nonionic surfactants, preference is given to fatty alcohol ethoxylates (alkyl polyethylene glycols); alkylphenol polyethylene glycols; alkyl mercaptan polyethylene glycols; fatty amine ethoxylates (alkylaminopolyethylene glycols); fatty acid ethoxylates (acyl polyethylene glycols); polypropylene glycol ethoxylates (®Pluronics); fatty acid amide polyethylene glycols; N-alkyl-, N-alkoxypolyhydroxy fatty acid amide, preferably fatty acid N-methylglucamides and sucrose esters; polyglycol ethers; alkyl polyglycosides; and/or phosphoric esters (mono-, di- and triphosphoric esters ethoxylated and nonethoxylated).

The proportion by weight of the nonionic surfactants, based on the finished compositions, is preferably 1 to 20% by weight, particularly preferably 2 to 10% by weight, especially preferably 3 to 7% by weight.

As amphoteric surfactants, preference is given to N-(C₁₂-C₁₈)alkyl-β-aminopropionates and N-(C₁₂-C₁₈)alkyl-β-iminodipropionates as alkali metal and mono-, di- and trialkylammonium salts; N-acylaminoalkyl-N, N-dimethylacetobetaine, preferably N-(C₈-C₁₈)acylaminopropyl-N,N-dimethylacetobetaine; (C₁₂-C₁₈)-alkyldimethylsulfopropylbetaine; amphoteric surfactants based on imidazoline (trade names Miranol®, Steinapon®), preferably the sodium salt of 1-(β-carboxymethyloxyethyl)-1-(carboxymethyl)-2-laurylimidazolinium; amine oxides, e.g. (C₁₂-C₁₈)alkyldimethylamine oxides; and/or fatty acid amidoalkyldimethylamine oxides.

The proportion by weight of the amphoteric surfactants, based on the finished compositions, is preferably 0.5 to 20% by weight, particularly preferably 1 to 10% by weight.

Furthermore, foam-booster cosurfactants from the group consisting of alkylbetaines, alkylamidobetaines, aminopropionates, aminoglycinates, imidazoliniumbetaines, sulfobetaines, amine oxides, fatty acid alkanolamides and polyhydroxyamides can be used in the compositions according to the invention.

Particularly preferred surfactants are lauryl sulfate, laureth sulfate, cocoamidipropylbetaine, sodium cocoyl glutamate, di-sodium laureth sulfosuccinate and/or coconut fatty acid diethanolamide.

In addition, the compositions can comprise superfatting agents, fats, waxes, stabilizers, biogenic active ingredients, glycerol, preservatives, pearlizing agents, dyes and fragrances, solvents, opacifiers, thickeners, dispersants, protein derivatives (e.g. gelatine), collagen hydrolyzates, natural- and synthetic-based polypeptides, egg yolk, lecithin, lanolin, lanolin derivatives, fatty alcohols, silicones, deodorizing agents, substances with a keratolytic and keratoplastic effect, enzymes, carrier substances, moisturizing substances and/or antimicrobial agents.

As superfatting agents, preference is given to polyethoxylated lanolin derivatives, lecithin derivatives, polyol fatty acid esters, monoglycerides and fatty acid alkanolamides, the latter also being suitable as foam stabilizers.

Preferred fats are glycerides; suitable waxes are, inter alia, beeswax, paraffin wax or microcrystalline waxes, optionally in combination with hydrophilic waxes, e.g. cetylstearyl alcohol.

Stabilizers which can be used are metal salts of fatty acids such as, for example, magnesium stearate, aluminum stearate and/or zinc stearate.

Biogenic active ingredients are, for example, understood as meaning plant extracts and vitamin complexes.

Suitable preservatives are, for example, phenoxyethanol, formaldehyde solution, parabens, pentanediol and sorbic acid.

Dyes which can be used are the substances approved and suitable for cosmetic purposes.

As thickeners and dispersants, preference is given to sodium chloride, potassium chloride, ammonium chloride, sodium sulfate, fatty acid alkylolamides, cellulose derivatives, for example hydroxyethylcellulose, guar gum, polyvinyl alcohol, polyvinylpyrrolidone, hydroxypropyl guar gum, starch and starch derivatives, and natural gums, carboxyvinyl polymers (e.g. the Carbopol® grades 934, 940, 941, 956, 980, 981, 1342 and 1382).

Particularly suitable as thickeners and dispersants are ethylene glycol esters of fatty acids having 14 to 22, particularly preferably 16 to 22, carbon atoms, in particular mono- and diethylene glycol stearate. Preference is also given to stearin monoethanolamide, stearin diethanolamide, stearin isopropanolamide, stearin monoethanolamide stearate, stearyl stearate, cetyl palmitate, glyceryl stearate, stearamide diethanolamide distearate, stearamide monoethanolamide stearate, N,N-dihydrocarbyl-(C₁₂-C₂₂)-, preferably (C₁₆-C₁₈), -amidobenzoic acid and soluble salts thereof and/or N,N-di(C₁₆-C₁₈) amidobenzoic acid and derivatives thereof.

The thickeners and dispersants are preferably used in concentrations, based on the finished compositions, of from 0.5 to 10% by weight, particularly preferably 0.5 to 5% by weight, especially preferably 1 to 4% by weight.

The desired viscosity of the compositions can be established by adding water and/or organic solvents or by adding a combination of organic solvents and thickeners. Suitable organic solvents are, in principle, all mono- or polyhydric alcohols and ethoxylated alcohols. Preference is given to alcohols with 1 to 4 carbon atoms, such as ethanol, propanol, isopropanol, n-butanol and isobutanol, glycerol and mixtures of said alcohols.

Further suitable solvents are, for example, triacetin (glycerol triacetate) and 1-methoxy-2-propanol.

The compositions according to the invention comprise, based on the finished compositions, the alcohols in amounts of from 0.1 to 50% by weight.

Suitable carrier materials are preferably vegetable oils, natural and hydrogenated oils, waxes, fats, water, alcohols, polyols, glycerol, glycerides, liquid paraffins, liquid fatty alcohols, sterol, cellulose and cellulose derivatives.

Fungicidal active ingredients which may be used are ketoconazole, oxiconazole, terbinafine, bifonazole, butoconazole, cloconazole, clotrimazole, econazole, enilconazole, fenticonazole, isoconazole, miconazole, sulconazole, tioconazole, fluconazole, itraconazole, terconazole, naftifine and terbinafine, Zn -pyrethione and octopyrox.

Suitable deodorizing substances may be allantoin and bisabolol, preferably in amounts by weight of from 0.0001 to 10% by weight.

As cationic polymers, preference is given to cationic cellulose derivatives, cationic starch, copolymers of diallylammonium salts and acrylamides, quaternized vinylpyrrolidone/vinylimidazole polymers, condensation products of polyglycols and amines, quaternized collagen polypeptides, quaternized wheat polypeptides, polyethyleneimine, cationic silicone polymers, such as, for example, amidomethicones, copolymers of adipic acid and dimethylaminohydroxypropyl-diethylenetriamine, polyaminopolyamide and cationic chitin derivatives, such as, for example, chitosan.

Suitable silicone compounds are, for example, dimethylpolysiloxane, methyl-phenylpolysiloxanes, cyclic silicones, and amino-, fatty acid-, alcohol-, polyether-, epoxy-, fluorine- and/or alkyl-modified silicone compounds, and also polyalkylsiloxanes, polyalkylarylsiloxanes, polyether siloxane copolymers, as described in U.S. Pat. No. 5,104,645 and the specifications cited therein which may either be in liquid form or else in resin form at room temperature.

The compositions according to the invention can be mixed with conventional ceramides, pseudoceramides, fatty acid N-alkylpolyhydroxyalkylamides, cholesterol, cholesterol fatty acid esters, fatty acids, triglycerides, cerebrosides, phospholipids and similar substances.

Suitable pearlescence-imparting compounds are fatty acid monoalkanolamides; fatty acid dialkanolamides; monoesters and diesters of alkylene glycol, in particular those of ethylene glycol, propylene glycol or oligomers thereof and higher fatty acids, e.g. palmitic acid, stearic acid, behenic acid or mixtures thereof; mono- or diesters of alkylene glycols with fatty acids; fatty acids and metal salts thereof; monoesters or polyesters of glycerol with carboxylic acids; and/or ketosulfones.

As pearlescence-imparting component, particular preference is given to ethylene glycol distearate and polyethylene glycol distearate with 3 glycol units.

As moisturizing substance, preference is given to isopropyl palmitate, glycerol and/or sorbitol, preferably in amounts by weight, based on the finished compositions, of from 0.1 to 50% by weight.

The invention likewise provides for the use of polyethylene glycols of the formula H(OCH₂CH₂)ₙOH, in which n is an integer between 150 and 900, as foam regulator in cosmetic compositions.

In this connection, the effect as foam regulator is understood as meaning that the creaminess, the stability, the fineness and the ability of the foam to be washed off of the cosmetic compositions can be controlled via the polyethylene glycols. The higher the molar mass and the amount of polyethylene glycol used, the more creamy and finer the foam which precipitates out. The ability to be washed off is better with a low molar mass than with a high molar mass.

The invention likewise provides for the use of polyethylene glycols of the formula H(OCH₂CH₂)ₙOH, in which n is an integer between 150 and 900, in cosmetic compositions for improving the feel of the skin and feel of the hair.

The invention further provides for a method for improving the combability, in particular the wet combability, of hair, comprising the treatment of the hair with a cosmetic composition comprising at least one polyethylene glycol of the formula H(OCH₂CH₂)ₙOH, in which n is an integer between 150 and 900. Improved combability is understood here as meaning that the use of the polyethylene glycols reduces the mechanical resistance to combing.

The cosmetic compositions are preferably hair-treatment compositions, particularly preferably shampoos and rinses, which are applied in accordance with the generally known procedures.

For the uses according to the invention as foam regulator and for improving the feel of the skin, and also the method according to the invention for improving the combability of the hair, polyethylene glycols of the formula H(OCH₂CH₂)ₙOH, in which n is an integer between 180 and 800, preferably between 240 and 800, particularly preferably between 300 and 800, are particularly suitable.

The examples below serve to illustrate the subject-matter of the invention in more detail, without limiting it thereto.

### EXAMPLES

Testing of foaming behavior, feel of the skin, feel of the hair and combability of the hair:

In a double-blind sensory panel test (10 subjects), surfactant formulations of ether sulfate/betaine (ratio of active content 7:3) with polyglycol 35,000 S (test formulation) and without polyglycol 35,000 S (standard formulation) were tested. Evaluation was made on a scale from −1.5 (maximum negative score) to +1.5 (maximum positive score).

| formulation | Standard | | Test | |
|---|---|---|---|---|
| Formulation of the surfactant base: | | | | |
| Sodium Laureth Sulfate | 48.1% | by wt. | 48.1% | by wt. |
| (27% active) | | | | |
| Cocamidopropylbetaine | 6.7% | by wt. | 6.7% | by wt. |
| (30% active) | | | | |
| Polyglycol 35 000 S | — | | 0.5% | by wt. |
| Demin. water | ad 100 | | ad 100 | |
| NaCl | 1.8% | by wt | 2.1% | by wt. |
| Evaluation: | | | | |
| 1) Foaming behavior: | | | | |

| | | |
|---|---|---|
| Onset of foaming behavior | 0.4 | 0.5 |
| (rapid/slow) | | |
| Ability to be distributed | 0.6 | 0.8 |
| (easy/difficult) | | |
| Amount of foam (large/small) | 0.1 | 0.3 |
| Foam bubble size (fine/coarse) | 0.4 | 0.7 |
| Foam creaminess (creamy/watery) | 0.2 | 0.7 |
| Ability of the foam to be washed | 0.5 | 0.6 |
| off (easy/difficult) | | |
| 2) Feel of the skin: | | |
| Feel of the skin wet | 0 | 0.5 |
| (smooth/rough) | | |
| Feel of the skin damp | −0.6 | 0.2 |
| (smooth/rough) | | |
| Care sensation dried skin (soft/dry) | −0.2 | 0.7 |
| Smoothness of the dried skin | −0.2 | 0.8 |
| (smooth/rough) | | |
| 3) Feel of the hair: | | |
| Feel of the hair (sleek/harsh) | 0 | 0.3 |
| 4) Combability of the hair: | | |
| Combability dry (good/poor) | −1 | 0.3 |
| Combability wet (good/poor) | −0.8 | 0.5 |

### Result

The test formulation with polyglycol 35,000 S received an overall score of +6.9 and thus came out significantly better than the standard formulation at −0.6 points. Particularly significant differences were observed for the creaminess of the foam, the feel of the skin (wet, damp and dry), the smoothness of the skin, and the wet combability.

### Formulation Example 1

### Shower Gel

| Components | | Amount in % by wt. |
|---|---|---|
| 1 | Carbopol ETD 2020 | 1.5 |
| 2 | Polyquaternium-10 | 0.3 |
| 3 | Glycerol | 2.0 |
| 4 | Polyglycol 35 000 S | 2.0 |
| 5 | Genagen LDA | 9.2 |
| 6 | Genagen CAB | 4.0 |
| 7 | Hostapon CLG | 4.8 |
| 8 | Citric acid | 0.5 |
| 9 | Methyldibromoglutaronitrile/phenoxyethanol | 0.05 |
| 10 | Perfume | 0.5 |
| 11 | Opacifyer 641 Opacifier | 0.8 |
| 12 | Demin. water | ad 100 |

### Preparation

Component 1 and 2 were introduced and dissolved in about 70° C. hot demin. water with stirring. One after the other, components 3, 4, 5, 6 and 7 were added with stirring and the pH was adjusted to pH 6.0 with citric acid. By adding components 9 and 10, the composition was preserved and perfumed. The opacifier 11 was then added.

### Formulation Example 2

### Shower Gel

| Components | | Amount in % by wt. |
|---|---|---|
| 1 | Carbopol ETD 2020 | 3.0 |
| 2 | Polyquaternium-10 | 0.3 |
| 3 | Polyglycol 20 000 S | 3.0 |
| 4 | Medialan LD | 2.0 |
| 5 | Genagen LAA | 7.2 |
| 6 | Genagen CAB | 4.0 |
| 7 | Hostapon KCG | 6.9 |
| 8 | Lactic acid | 0.5 |
| 9 | Preservative | q.s. |
| 10 | Perfume | q.s. |
| 11 | Genapol TSM | 1.0 |
| 12 | Demin. water | ad 100 |

### Preparation

Components 1 and 2 were initially introduced and dissolved in about 70° C. hot demin. water with stirring. One after the other, components 3, 4, 5, 6 and 7 were added with stirring and the pH was adjusted to pH 6.0 with lactic acid. By adding components 9 and 10, the composition was preserved and perfumed. The silky luster agent 11 was then added.

### Formulation Example 3

### Hair Shampoo

| Components | | Amount in % by wt. |
|---|---|---|
| 1 | Genapol LRO liquid | 11.10 |
| 2 | Fragrance | 0.30 |
| 3 | Demin. water | ad 100 |
| 4 | Genagen CAB | 24.00 |
| 5 | Genagen LAA | 11.60 |
| 6 | Polyglycol 35 000 S | 2.00 |
| 7 | Citric acid (50% strength in water) | 1.20 |
| 8 | Dye solution | q.s. |
| 9 | Preservative | q.s. |

### Preparation

Component 1 was initially introduced, then the other components were stirred in in the order given.

### Formation Example 4

### Cream Rinse

| Components | | Amount in % by wt. |
|---|---|---|
| 1 | Genamin DSAC | 1.50 |
| 2 | Hostacerin T-3 | 1.50 |
| 3 | Cetyl alcohol | 2.50 |
| 4 | Paraffin oil, high-viscosity | 1.00 |
| 5 | Genamin KSL | 2.00 |
| 6 | Demin. water | 91.20 |
| 7 | Polyglycol 20 000 S | 2.00 |
| 8 | Preservative | q.s. |
| 9 | Perfume oil | 0.30 |
| 10 | Dye solution | q.s. |
| 11 | Citric acid | q.s. |

### Preparation

Components 1 to 4 were melted at about 75° C. Components 5 to 8 were then heated to about 75° C. and added with stirring. The mixture was then stirred until cold. Then, at about 35° C., components 9 and 10 were stirred in, and finally the pH was adjusted to pH 4 with citric acid.

### List of Products Used

| | | |
|---|---|---|
| Carbopol ® ETD | (Goodrich) | Polyacrylic acid, crosslinked |
| 2020 | | cation. modified cellulose ether |
| Polyquaternium-10 | | |
| Polyglycol 20 000 S | (Clariant GmbH) | PEG-350, polyethylene glycol |
| | | with average molar mass 20 000 |
| | | g/mol |
| Polyglycol 35 000 S | (Clariant GmbH) | PEG-800, polyethylene glycol |
| | | with average molar mass 35 000 |
| | | g/mol |
| Genagen ® LDA | (Clariant GmbH) | Lauryl amphodiacetate, Na salt |
| Genagen ® LAA | (Clariant GmbH) | Laurylamphoacetate, Na salt |
| Genagen ® CAB | (Clariant GmbH) | Cocoamidopropylbetaine |
| Hostapon ® CLG | (Clariant GmbH) | Sodium lauryl glutamate |
| Hostapon ® KCG | (Clariant GmbH) | Sodium cocoyl glutamate |
| Medialan ® LD | (Clariant GmbH) | Sodium lauroyl sarcosinate |
| Genapol ® TSM | (Clariant GmbH) | PEG-3 distearate, sodium |
| | | laureth sulfate |
| Opacyfier ® 641 | (Lytron) | Sodium styrene/acrylates |
| | | copolymer |
| Genapol ® LRO | (Clariant GmbH) | Sodium laureth sulfate |
| Genamin ® KSL | (Clariant GmbH) | PEG-5 Stearyl ammonium |
| | | lactate |
| Hostacerin ® T-3 | (Clariant GmbH) | Ceteareth-3 |
| Genamin ® DSAC | (Clariant GmbH) | Distearyldimonium chloride |

1. A cosmetic composition comprising polyethylene glycols of the formula H(OCH₂CH₂)ₙOH, in which n is an integer between 150 and 900.

2. The cosmetic composition as claimed in claim 1, wherein n is an integer between 180 and 800.

3. The cosmetic composition as claimed in claim 1, wherein n is an integer between 240 and 800.

4. The cosmetic composition as claimed in claim 1, wherein n is an integer between 300 and 800.

5. The cosmetic composition as claimed in claim 1, wherein the finished compositions comprise the polyethylene glycols in an amount of from 0.1 to 50% by weight.

6. The cosmetic composition as claimed in claim 1, wherein the finished compositions comprise the polyethylene glycols in an amount of from 0.5 to 10% by weight.

7. The cosmetic composition as claimed in claim 1, wherein the finished compositions comprise the polyethylene glycols in an amount of from 1 to 4% by weight.

8. The cosmetic composition as claimed in claim 1, further comprising cationic guar gum polymers.

9. The cosmetic composition as claimed in claim 8, wherein the cationic guar gum polymers are guar hydroxypropyltrimethylammonium chlorides.

10. The cosmetic composition as claimed in claim 8, wherein the amount of cationic guar gum polymers, based on the finished compositions, is 0.01 to 1.0% by weight.

11. The cosmetic composition as claimed in claim 8, wherein the amount of cationic guar gum polymers, based on the finished compositions, is 0.02 to 0.4% by weight.

12. A hair or skin treatment selected from the group consisting of a shampoo, a hair shampoo, a rinse, a conditioner, a cream rinse, a cream, a ointment, gel, a shower gel, a shower preparation, and a foam bath comprising the cosmetic composition of claim 1.

13. A process for regulating foam in a cosmetic composition comprising adding polyethylene glycols of the formula H(OCH₂CH₂)ₙOH, in which n is an integer between 150 and 900, to said cosmetic composition.

14. The process of claim 13 wherein n is an integer between 180 and 800.

15. The process of claim 13 wherein n is an integer between 240 and 800.

16. The process of claim 13 wherein n is an integer between 300 and 800.

17. Process for improving the feel of the skin and feel of the hair in a cosmetic composition comprising adding polyethylene glycols of the formula H(OCH₂CH₂)ₙOH, in which n is an integer between 150 and 900, to said cosmetic composition.

18. The process of claim 17 wherein the cosmetic composition includes cationic guar gum polymers.

19. The process of claim 18 wherein the cosmetic composition comprises from 0.5 to 10 weight percent of said polyethylene glycols and from 0.01 to 10 weight percent of the cationic guar gum polymers.

20. The process of claim 17 wherein n is an integer between 180 and 800.

21. The process of claim 17 wherein n is an integer between 240 and 800.

22. The process of claim 17 wherein n is an integer between 300 and 800.

23. A process for improving combability of wet hair comprising treating the wet hair with a cosmetic composition comprising at least one polyethylene glycol of the formula H(OCH₂CH₂)ₙOH, in which n is an integer between 150 and 900.

24. The process of claim 23 wherein the cosmetic composition further comprises cationic guar gum polymers.

25. The process of claim 24 wherein the cationic guar gum ploymer comprises guar hydroxypropyltrimethylammonium chlorides.

26. The process of claim 23 wherein n is an integer between 180 and 800.

27. The process of claim 23 wherein n is an integer between 240 and 800.

28. The process of claim 23 wherein n is an integer between 300 and 800.
